(19) United States
(12) Patent Application Publication
(10) Pub. No.: US 2015/0366697 A1
(43) Pub. Date: Dec. 24, 2015

(54) ORTHOTIC BACK BRACE

(71) Applicant: Retrainer Inc., Las Vegas, NV (US)

(72) Inventor: Edward Ronald Dreske, Taylorsville, UT (US)

(21) Appl. No.: 14/312,063

(22) Filed: Jun. 23, 2014

(51) Int. Cl. A61F 5/02

(52) U.S. Cl. CPC A61F 5/02

(57) ABSTRACT

A brace for supporting a patient's spine. The brace includes a band having a central back portion, a left abdominal portion, and a right abdominal portion. The band tapers over the left and right portions from wider rearward ends to narrower forward ends. The abdominal portions have complementary fasteners adjacent the corresponding forward ends for joining the portions. The brace has a rigid dorsal panel attached to the central back portion of the band and extending upward from a lower end aligned with the lower edge of band, upward to an upper end. The upper end and the lower end are spaced by a distance corresponding to a distance between a sacrococcygeal junction and a position below the patient's third thoracic vertebra. The brace also includes left and right rigid vertical stays, left and right shoulder straps, and left and right closure straps.

### BACKGROUND

The present invention generally relates to an orthotic back brace, and more particularly, to a thoracic-lumbar-sacral orthotic brace.

Various braces are used for supporting a patient's spine when recovering from injury or surgery, or to prevent progression of deformity. For example, some braces have combinations of a thick, hard-shell back plate, side plates, and an abdominal plate held in place around the patient with wide elastic bands or straps. Further, the thick plates extend from below the patient's waist to a position below the patient's armpits or aligned with the breastbone. The thickness of the plates interferes with patient movement other than movement of the spine. Further, the plates protrude significantly, causing difficulty in fitting outerwear and a generally unsightly appearance.

Thoracic-lumbar-sacral orthotic (TLSO) braces generally extend from the sacrum to a position above a lower end of the scapulae (i.e., above the seventh thoracic vertebra) and are used to support and stabilize the patient's trunk. Such support may be needed following paralysis, spinal fusion, or surgery, as well as to prevent progression of spinal deformity (e.g., moderate scoliosis). Even though some of these braces have thinner plates, the plates are still broad and rigid. Typically, the braces have a broad back plate extending from below the patient's waist to a position near the patient's neck or scapula. The braces also include a broad abdominal plate extending from below the patient's waist to a position below the patient's breastbone. These plates are held in place with broad elastic bands or straps. Scaffolding frequently extends between the top of the abdominal plate and the patient's shoulders where the scaffolding joins shoulder straps that extend to the back plate. Although effective, these braces, like the hard shell braces, are extraordinarily confining and unsightly. In some instances, these characteristics have caused reduced patient compliance, sometimes causing longer recovery or even re-injury.

### SUMMARY

In one aspect, the present invention includes a brace for supporting a patient's spine. The brace comprises a flexible band having a central back portion, a left abdominal portion extending from a left side of the central back portion, and a right abdominal portion extending from a right side of the central back portion. The band has an inner face facing the patient in use and an outer face opposite the inner face. The band includes a lower edge extending from the left abdominal portion, through the central back portion, to the right abdominal portion, and an upper edge opposite the lower edge. The band tapers over the left and right abdominal portions from relatively wider rearward ends to relatively narrower forward ends. The left and right abdominal portions have complementary fasteners adjacent the corresponding forward ends for joining the left and right abdominal portions when at least partially overlap the patient's abdomen in use. In addition, the brace includes a rigid dorsal panel attached to the central back portion of the band. The panel extends upward from a lower end aligned with the lower edge of band, upward past the upper edge of the central back portion to an upper end. The upper end and lower end are spaced by a distance corresponding to a distance between a sacrococcygeal junction of the patient and a position above the patient's seventh thoracic vertebra and below the patient's third thoracic vertebra. Further, the brace comprises left and right rigid vertical stays attached to the band on corresponding opposite sides of the dorsal panel. The brace also includes left and right shoulder straps extending from opposite corners of the upper end of the dorsal panel to the outer face of the band on corresponding sides of the central back portion. The straps are sized for accepting corresponding shoulders of the patient in use. Lastly, the left and right closure straps extend from the outer face of the band on corresponding sides of the central back portion. The closure straps are sized to overlap the left and right abdominal portions of the band when at least partially overlapping the patient's abdomen in use. The closure straps have complementary fasteners for joining the left and right closure straps when overlapping the left and right abdominal portions of the band in use.

In another aspect, the present invention includes a brace for supporting a patient's spine. The brace comprises a flexible band having a central back portion, a left abdominal portion extending from a left side of the central back portion, and a right abdominal portion extending from a right side of the central back portion. The band has an inner face facing the patient in use and an outer face opposite the inner face. The band includes a lower edge and an upper edge opposite the lower edge. The band tapers over the left and right abdominal portions from relatively wider rearward ends to relatively narrower forward ends. The left and right abdominal portions have complementary fasteners adjacent the corresponding forward ends for joining the left and right abdominal portions when at least partially overlapping the patient's abdomen in use. In addition, the brace includes a rigid dorsal panel attached to the central back portion of the band. The dorsal panel extends upward from a lower end aligned with the lower edge of band, upward past the upper edge of the central back portion to an upper end. The upper end and the lower end are spaced by a distance corresponding to a distance between a sacrococcygeal junction of the patient and a position above the patient's seventh thoracic vertebra and below the patient's third thoracic vertebra. Further, the brace includes left and right rigid vertical stays attached to the band on corresponding opposite sides of the dorsal panel. The brace also has left and right shoulder straps extending from opposite corners of the upper end of the dorsal panel to the outer face of the band. The straps are sized for accepting corresponding shoulders of the patient in use. Moreover, the brace comprises left and right closure straps extending from the outer face of the band. The closure straps are sized to overlap the left and right abdominal portions of the band when at least partially overlapping the patient's abdomen in use. The closure straps have complementary fasteners for joining the left and right closure straps when overlapping the left and right abdominal portions of the band in use. The brace is substantially free of rigid structure above the left and right abdominal portions and between the left and right shoulder straps.

In yet another aspect, the present invention includes a brace for supporting a patient's spine. The brace comprises a flexible band having a central back portion, a left abdominal portion extending from a left side of the central back portion, and a right abdominal portion extending from a right side of the central back portion. The band has an inner face facing the patient in use and an outer face opposite the inner face. The band includes a lower edge and an upper edge opposite the lower edge. The band tapers over the left and right abdominal portions from relatively wider rearward ends to relatively narrower forward ends. The left and right abdominal portions have complementary fasteners adjacent the corresponding forward ends for joining the left and right abdominal portions when at least partially overlapping the patient's abdomen in use. In addition, the brace in a rigid dorsal panel attached to the central back portion of the band and extending upward from a lower end aligned with the lower edge of band, upward past the upper edge of the central back portion to an upper end. The upper end and the lower end are spaced by a distance corresponding to a distance between a sacrococcygeal junction of the patient and a position immediately below the scapular spine of the patient. The brace also includes left and right rigid vertical stays attached to the band on corresponding opposite sides of the dorsal panel. Further, the brace has left and right shoulder straps extending from opposite corners of the upper end of the dorsal panel to the outer face of the band on corresponding sides of the central back portion. The straps are sized for accepting corresponding shoulders of the patient in use. In addition, the brace comprises left and right closure straps extending from the outer face of the band on corresponding sides of the central back portion. The closure straps are sized to overlap the left and right abdominal portions of the band when at least partially overlapping the patient's abdomen in use. The closure straps have complementary fasteners for joining the left and right closure straps when overlapping the left and right abdominal portions of the band in use.

Other aspects of the present invention will be apparent in view of the following description and claims.

### BRIEF DESCRIPTION OF THE DRAWINGS

FIG. 1 is a front perspective of a brace of the present invention;

FIG. 2 is a rear perspective of the brace;

FIG. 3 is a fragmentary front elevation of the brace opened flat to show the structure thereof; and

FIG. 4 is a fragmentary rear elevation of the brace opened flat.

Corresponding reference characters indicate corresponding parts throughout the drawings.

### DETAILED DESCRIPTION OF THE DRAWINGS

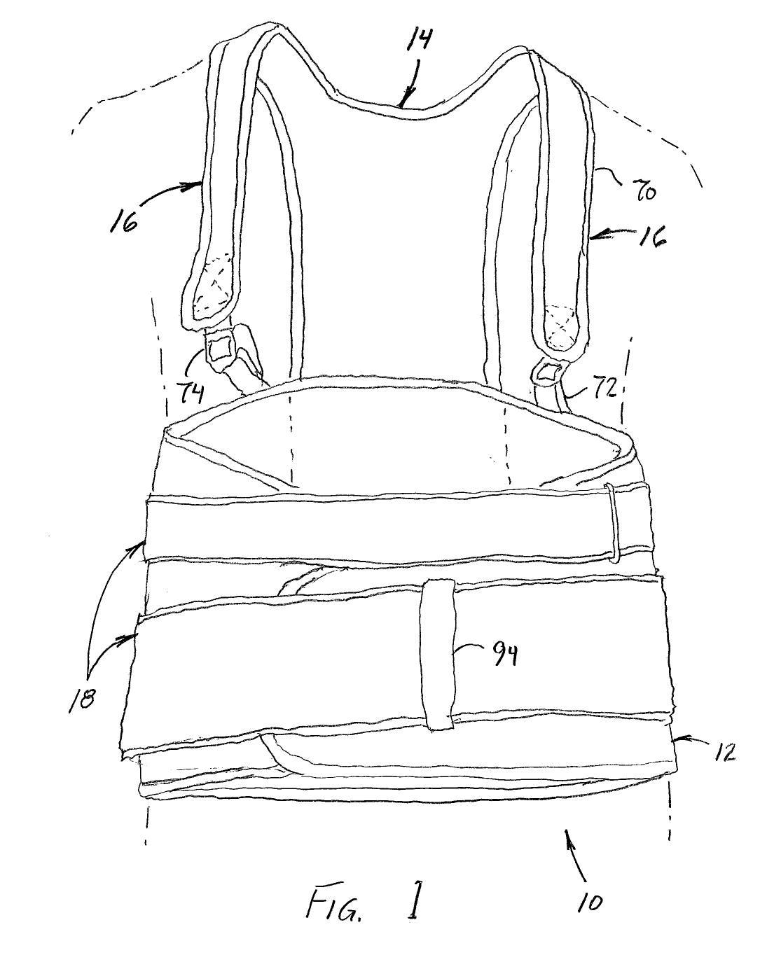

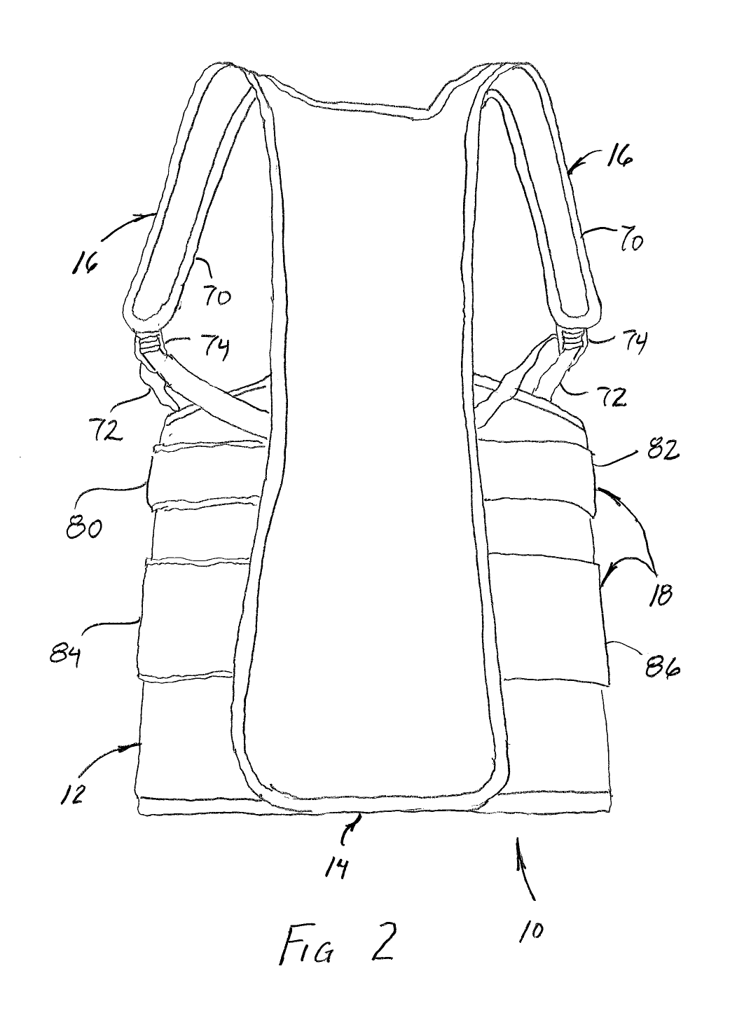

Referring to FIGS. 1 and 2, a thoracic-lumbar-sacral orthotic brace incorporating one embodiment of the present invention is designated in its entirety by the reference number 10. The brace 10 generally comprises a broad, flexible, and inelastic band (generally designated by 12) for providing abdominal compression and posterior upper thoracic extension, a rigid dorsal panel or backplane (generally designated by 14) for stabilizing the upper thoracic and lumbar spine, left and right shoulder straps (generally designated by 16) for maintaining the thoracic spine in position on the dorsal panel, and left and right closure straps (generally designated by 18) for retaining the band in position on a patient (indicated in phantom in FIG. 1).

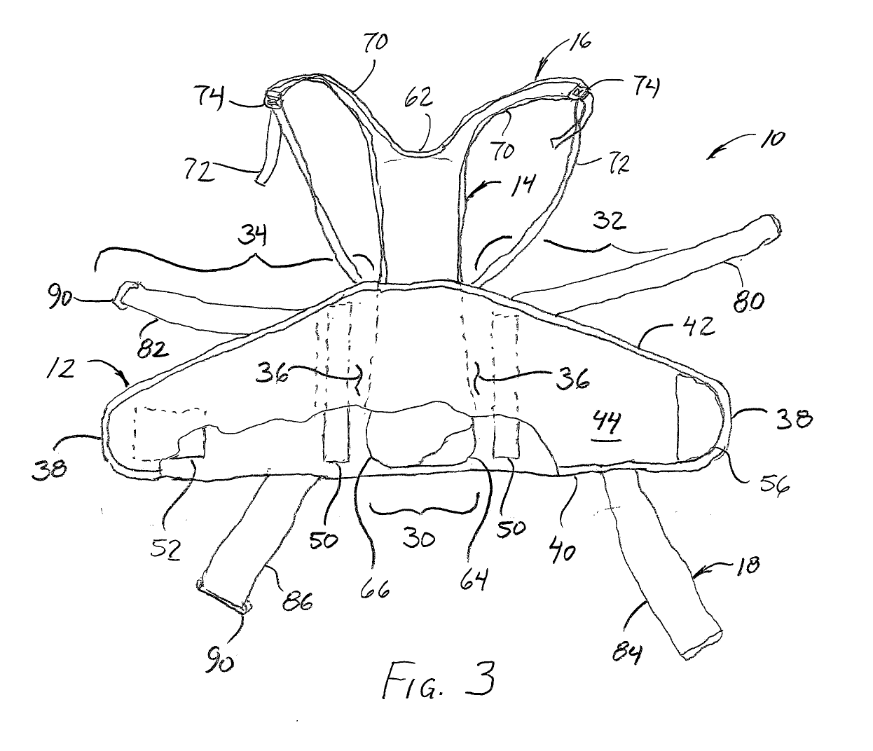

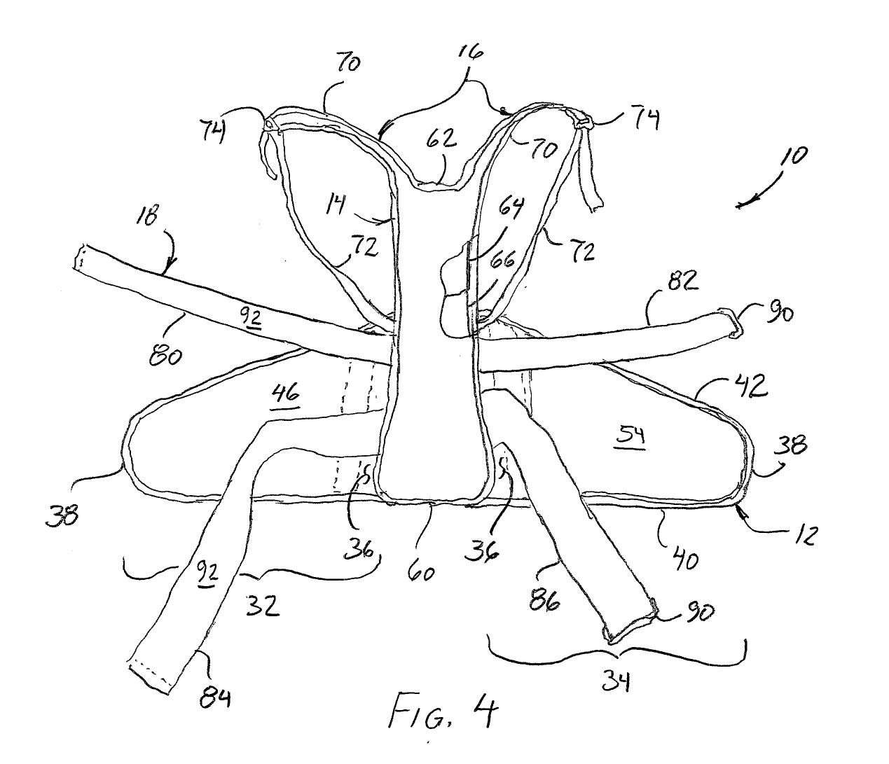

As shown in FIGS. 3 and 4, the band 12 has a central back portion 30, a left abdominal portion or wing 32 extending from the left side of the back portion, and a right abdominal portion or wing 34 extending from the right side of the back portion. The left and right abdominal portions 32, 34 taper as shown so that their rearward ends 36 adjacent opposite sides of the back portion are wider than the forward ends 38. Further, the central back portion 30 and the abdominal portions 32, 34 of the band 12 have a generally straight, horizontal lower edge 40. The abdominal portions 32, 34 have slanted or oblique upper edges 42, resulting in the tapered characteristic described above. Although the band may be made of other materials without departing from the scope of the present invention, in one embodiment the band 12 includes a 500 denier polyester fabric having an anti-bacterial and anti-fungal coating forming an inner surface 44 (FIG. 3) that faces the patient during use and a 1680 denier ballistic nylon fabric forming an outer surface 46 (FIG. 4). In this embodiment, one inch binding tape surrounds the perimeter of the band 12.

A narrow, rigid stay 50 is sewn between the fabrics forming the inner and outer surfaces 44, 46, respectively, on each side of the central back portion 30 and the rigid dorsal panel 14. The rigid stays 50 extend vertically between the upper and lower edges 42, 40, respectively, of the band 12. Although the stays may be made of other materials without departing from the scope of the present invention, in one embodiment the stays 50 are made from 1/16 inch thick styrene sheet. A rigid, rectangular abdominal panel 52 is sewn between the fabrics forming the inner and outer surfaces 40, 42 at the forward end 38 of the right abdominal portion 34 for overlying a patient's abdomen in use. Although the abdominal panel may be made of other materials without departing from the scope of the present invention, in one embodiment the abdominal panel 52 is made from 1/16 inch thick styrene sheet. The outer surface 42 of the right abdominal portion 34 of the band 12 is covered with pile material constituting loop fabric 54 over an area extending from adjacent the stay 50 to the forward end 38. The inner surface 40 of the left abdominal portion 32 of the band 12 is covered with hook fabric 56 adjacent the forward end 38. The hook and loop fabrics form complementary fasteners for joining the left and right abdominal portions 32, 34 when they overlap the patient's abdomen in use.

As further shown in FIG. 4, the rigid dorsal panel 14 is attached to the central back portion 30 of the band 12. The dorsal panel 14 extends upward from a lower end 60 that is aligned with the lower edge 40 of band 12. The panel 14 extends upward past the upper edge 42 of the central back portion 30 to an upper end 62. In use, the lower end 60 is positioned adjacent the sacrococcygeal junction, and the upper end 62 is positioned just below the scapular spine (i.e., below the third thoracic vertebra) of the patient. Thus, the upper end 62 and the lower end 60 are spaced by a distance corresponding to a distance between a sacrococcygeal junction of the patient and a position above the patient's seventh thoracic vertebra and below the patient's third thoracic vertebra. Although the dorsal panel may be made of other materials without departing from the scope of the present invention, in one embodiment the dorsal panel 14 is formed from ⅛ inch thick ABS plastic sheet 64 having ¼ inch thick closed cell foam 66 overlying its inner surface. Further, in this embodiment the plastic sheet 64 and foam 66 are covered with a shell formed from ballistic nylon fabric and polyester fabric joined with binding tape as described above with respect to the band 12.

FIGS. 3 and 4 illustrate that the left and right shoulder straps 16 extend obliquely upward from opposite corners of the upper end 62 of the dorsal panel 14. A rearward portion 70 of each shoulder strap 16 is unitary with the ballistic nylon and polyester shell covering the dorsal panel 14. Further, padding is provided in the rearward portion 70 of each shoulder strap 16 for patient comfort. Although the padding may be made of other materials without departing from the scope of the present invention, in one embodiment the padding is ¼ inch thick closed cell foam. It is envisioned this foam padding may be separate from or unitary with the foam 66 of the dorsal panel 14. A forward portion 72 of each shoulder strap 16 extends from the rearward portion 72 the outer surface or face 42 of the band 12 on corresponding sides of the central back portion 30. Although the forward portions may be made of other material without departing from the scope of the present invention, in one embodiment the forward portions 72 are made from one inch wide polyester webbing. Adjustable fasteners 74 join the forward and rearward portions 72, 70, respectively, for adjusting the overall length of the shoulder straps 16. Thus, the shoulder straps 16 may be sized to accept the patient's shoulders and adjusted to maintain the position of the brace 10 on the patient. Although other adjustable fasteners may be used without departing from the scope of the present invention, in one embodiment the adjustable fasteners 74 are one inch wide plastic ladder sliders sewn to the forward ends of the rearward portions 70. The forward portions 72 in this embodiment are threaded through the ladder sliders in a conventional manner.

As illustrated in FIGS. 3 & 4, left and right upper closure straps 80, 82, respectively, and left and right lower closure straps 84, 86, respectively, extend from the outer surface or face 42 of the band 12 on corresponding sides of the central back portion 30. Each pair of closure straps 80, 82 and 84, 86 are sized to overlap the left and right abdominal portions 32, 34 of the band 12 when those portions overlap the patient's abdomen in use. In one embodiment, the free end of the right closure straps 82, 86 have ladder locks 90 sized for receiving the corresponding left closure strap 80, 84. Further, in this embodiment the opposite faces of the left closure straps 80, 84 have complementary hook and loop fabric fasteners 92. As will be appreciated by those skilled in the art, the left closure straps 80, 84 are threaded through the corresponding ladder locks 90 on the right closure straps 82, 86 and fastened upon itself to hold the abdominal portions 32, 34 of the band 12 in position on the patient. Although the upper and lower closure straps may be made of other materials without departing from the scope of the present invention, in one embodiment the upper closure straps 80, 82 are made of two inch wide polyester webbing and the lower closure straps 84, 86 are made of four inch wide polyester webbing. Further, a fabric keeper 94 (FIG. 1) may be provided on one or both left closure straps 80, 82 to keep a tag end of the straps in position.

In order to use the brace 10, the patient's arms are inserted through loops created by the shoulder straps 16. The shoulder straps 16 are then positioned over the patient's shoulders. The band 12 is fastened about the patient's waist, and the upper and lower closure straps 80-86 are wrapped around the band and fastened. Any needed adjustments are then made to the band 12 and closure straps 80-86. Tails of the front portions 72 of the shoulder straps 16 are pulled downward to adjust the length of the shoulder straps to make final adjustments.

As will be appreciated by those skilled in the art, the brace 10 described above is substantially free of rigid structure such as scaffolding above the left and right abdominal portions 32, 34 of the band 12, as well as between the left and right shoulder straps 16. Even without the scaffolding or hard shells, the brace 10 provides abdominal compression and posterior upper thoracic extension with stabilization of the upper thoracic and lumbar regions. Accordingly, the brace 10 comfortably accommodate both men and women. And, the brace is light weight compared to braces incorporating scaffolding and/or hard shells. Further, the adjustable hook and loop fabric closures enable the brace 10 to be configured for nearly any body type or shape.

Having described the invention in detail, it will be apparent that modifications and variations are possible without departing from the scope of the invention defined in the appended claims.

When introducing elements of the present invention or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A brace for supporting a patient's spine, said brace comprising:
a flexible band having a central back portion, a left abdominal portion extending from a left side of the central back portion, and a right abdominal portion extending from a right side of the central back portion, said band having an inner face facing the patient in use and an outer face opposite the inner face, the band including a lower edge extending from the left abdominal portion, through the central back portion, to the right abdominal portion, and an upper edge opposite the lower edge, the band tapering over the left and right abdominal portions from relatively wider rearward ends to relatively narrower forward ends, the left and right abdominal portions having complementary fasteners adjacent the corresponding forward ends for joining the left and right abdominal portions when at least partially overlapping the patient's abdomen in use;
a rigid dorsal panel attached to the central back portion of the band and extending upward from a lower end aligned with the lower edge of band, upward past the upper edge of the central back portion to an upper end, the upper end and the lower end being spaced by a distance corresponding to a distance between a sacrococcygeal junction of the patient and a position above the patient's seventh thoracic vertebra and below the patient's third thoracic vertebra;
left and right rigid vertical stays attached to the band on corresponding opposite sides of the dorsal panel;
left and right shoulder straps extending from opposite corners of the upper end of the dorsal panel to the outer face of the band on corresponding sides of the central back portion, the straps being sized for accepting corresponding shoulders of the patient in use; and
left and right closure straps extending from the outer face of the band on corresponding sides of the central back portion, the closure straps being sized to overlap the left and right abdominal portions of the band when at least partially overlapping the patient's abdomen in use, said closure straps having complementary fasteners for joining the left and right closure straps when overlapping the left and right abdominal portions of the band in use.

2. A brace as set forth in claim 1, wherein each of the shoulder straps has an adjustable length.

3. A brace as set forth in claim 2, wherein each of the shoulder straps includes a ladder slider for adjusting the length of the respective strap.

4. A brace as set forth in claim 1, wherein the complementary fasteners on the left and right closure straps comprise fabric hook and loop fasteners.

5. A brace as set forth in claim 4, wherein one of the left and right closure straps includes a ladder lock sized and shaped for accepting another of the left and right closure straps.

6. A brace as set forth in claim 1, further comprising closed cell foam positioned on an inner surface of the dorsal panel in use.

7. A brace as set forth in claim 1, further comprising a rigid abdominal panel positioned on at least one of the left and right abdominal portions for overlying a patient's abdomen in use.

8. A brace as set forth in claim 1, each of the shoulder straps comprise closed cell foam.

9. A brace as set forth in claim 1, wherein the flexible band and rigid dorsal panel include an anti-bacterial, anti-fungal coating.

10. A brace as set forth in claim 1, wherein said left and right closure straps constitute lower left and right closure straps; and
the brace further comprises upper left and right closure straps extending from the outer face of the band on corresponding sides of the central back portion, the upper closure straps being sized to overlap the left and right abdominal portions of the band when at least partially overlapping the patient's abdomen in use, said upper closure straps having complementary fasteners for joining the upper left and right closure straps when overlapping the left and right abdominal portions of the band in use.

11. A brace for supporting a patient's spine, said brace comprising:
a flexible band having a central back portion, a left abdominal portion extending from a left side of the central back portion, and a right abdominal portion extending from a right side of the central back portion, said band having an inner face facing the patient in use and an outer face opposite the inner face, the band including a lower edge and an upper edge opposite the lower edge, the band tapering over the left and right abdominal portions from relatively wider rearward ends to relatively narrower forward ends, the left and right abdominal portions having complementary fasteners adjacent the corresponding forward ends for joining the left and right abdominal portions when at least partially overlapping the patient's abdomen in use;
a rigid dorsal panel attached to the central back portion of the band and extending upward from a lower end aligned with the lower edge of band, upward past the upper edge of the central back portion to an upper end, the upper end and the lower end being spaced by a distance corresponding to a distance between a sacrococcygeal junction of the patient and a position above the patient's seventh thoracic vertebra and below the patient's third thoracic vertebra;
left and right rigid vertical stays attached to the band on corresponding opposite sides of the dorsal panel;
left and right shoulder straps extending from opposite corners of the upper end of the dorsal panel to the outer face of the band, the straps being sized for accepting corresponding shoulders of the patient in use; and
left and right closure straps extending from the outer face of the band, the closure straps being sized to overlap the left and right abdominal portions of the band when at least partially overlapping the patient's abdomen in use, said closure straps having complementary fasteners for joining the left and right closure straps when overlapping the left and right abdominal portions of the band in use;
wherein the brace is substantially free of rigid structure above the left and right abdominal portions and between the left and right shoulder straps.

12. A brace as set forth in claim 11, wherein each of the shoulder straps has an adjustable length.

13. A brace as set forth in claim 11, wherein:
the complementary fasteners on the left and right closure straps comprise fabric hook and loop fasteners; and
one of the left and right closure straps includes a ladder lock sized and shaped for accepting another of the left and right closure straps.

14. A brace as set forth in claim 11 further comprising a rigid abdominal panel positioned on at least one of the left and right abdominal portions for overlying a patient's abdomen in use.

15. A brace as set forth in claim 11, wherein said left and right closure straps constitute lower left and right closure straps; and
the brace further comprises upper left and right closure straps extending from the outer face of the band, the upper closure straps being sized to overlap the left and right abdominal portions of the band when at least partially overlapping the patient's abdomen in use, said upper closure straps having complementary fasteners for joining the upper left and right closure straps when overlapping the left and right abdominal portions of the band in use.

16. A brace for supporting a patient's spine, said brace comprising:
a flexible band having a central back portion, a left abdominal portion extending from a left side of the central back portion, and a right abdominal portion extending from a right side of the central back portion, said band having an inner face facing the patient in use and an outer face opposite the inner face, the band including a lower edge and an upper edge opposite the lower edge, the band tapering over the left and right abdominal portions from relatively wider rearward ends to relatively narrower forward ends, the left and right abdominal portions having complementary fasteners adjacent the corresponding forward ends for joining the left and right abdominal portions when at least partially overlapping the patient's abdomen in use;
a rigid dorsal panel attached to the central back portion of the band and extending upward from a lower end aligned with the lower edge of band, upward past the upper edge of the central back portion to an upper end, the upper end and the lower end being spaced by a distance corresponding to a distance between a sacrococcygeal junction of the patient and a position immediately below the scapular spine of the patient;
left and right rigid vertical stays attached to the band on corresponding opposite sides of the dorsal panel;
left and right shoulder straps extending from opposite corners of the upper end of the dorsal panel to the outer face of the band on corresponding sides of the central back portion, the straps being sized for accepting corresponding shoulders of the patient in use; and
left and right closure straps extending from the outer face of the band on corresponding sides of the central back portion, the closure straps being sized to overlap the left and right abdominal portions of the band when at least partially overlapping the patient's abdomen in use, said closure straps having complementary fasteners for joining the left and right closure straps when overlapping the left and right abdominal portions of the band in use.

17. A brace as set forth in claim 16, wherein each of the shoulder straps has an adjustable length.

18. A brace as set forth in claim 16, wherein:
the complementary fasteners on the left and right closure straps comprise fabric hook and loop fasteners; and
one of the left and right closure straps includes a ladder lock sized and shaped for accepting another of the left and right closure straps.

19. A brace as set forth in claim 16 further comprising a rigid abdominal panel positioned on at least one of the left and right abdominal portions for overlying a patient's abdomen in use.

20. A brace as set forth in claim 16, wherein said left and right closure straps constitute lower left and right closure straps; and
the brace further comprises upper left and right closure straps extending from the outer face of the band, the upper closure straps being sized to overlap the left and right abdominal portions of the band when at least partially overlapping the patient's abdomen in use, said upper closure straps having complementary fasteners for joining the upper left and right closure straps when overlapping the left and right abdominal portions of the band in use.
